(19) United States
(12) Patent Application Publication
(10) Pub. No.: US 2006/0015083 A1
(43) Pub. Date: Jan. 19, 2006

(54) Absorbent hydrogels

(76) Inventors: Hugh Semple Munro, Chipping Campden (GB); Richard Hoskins, Swindon (GB); Susana Sainz Garcia, Oxfordshire (GB)

Correspondence Address: Min Hsieh & Hack;c/o PortfolioIP, P O Box 52050, Minneapolis, MN, 55402, US

(21) Appl. No.: 10/507,322

(22) PCT Filed: Mar. 11, 2003
(86) PCT No.: PCT/GB03/01014

§ 371 Date: Jun. 21, 2005

(30) Foreign Application Priority Data
Mar. 11, 2002 (GB) 0205702.4
May 20, 2002 (GB) 0211529.3
Nov. 7, 2002 (GB) 0226066.9

(51) Int. Cl. A61F 13/15

(52) U.S. Cl. 604/367

(57) ABSTRACT

The invention provides absorbent hydrogel compositions and hydrogel-containing structures. Typically, a first portion of the novel materials comprises a porous flexible plasticised hydrophilic polymer matrix having an internal cellular structure, and a second portion is relatively non-porous, e.g. comprises a flexible plasticised hydrophilic polymer matrix having relatively continuous internal structure. Methods for the manufacture or such hydrogels and structures are provided, as well as uses thereof.

### FIELD OF THE INVENTION

The present invention relates to absorbent (porous) hydrogels, and more particularly to hydrogels suitable for use in wound and burn dressings and other applications where a relatively high speed of fluid uptake is required. The invention also relates to processes for the manufacture of the novel hydrogels, and to uses of the hydrogels.

The expressions “hydrogel” and “hydrogel compositions” used herein are not to be considered as limited to gels which contain water, but extend generally to all hydrophilic gels and gel compositions, including those containing organic non-polymeric components in the absence of water.

### BACKGROUND OF THE INVENTION

U.S. Pat. No. 5,750,585 (Park et al), the disclosure of which is incorporated herein by reference, describes certain superabsorbent hydrogel foams comprising a solid phase and a gas phase, in which the volume of the gas phase exceeds the volume of the solid phase. Such foams may generally be thought of as relatively light foams. The preferred density of the foams is stated to be between 0.015 and 0.5. Higher densities are stated to be undesirable as the swelling of the foam is slower (prior art, column 7, lines 35 to 46).

The prior art foams are stated to have potential utility as superabsorbents, oral drug delivery vehicles and gastric retention devices for diet control.

Hydrogel foams of polyacrylamide, polyvinylpyrrolidone, poly-(2-hydroxyethyl-methacrylate) or poly-(2-hydroxypropyl-methacrylate) are specifically mentioned.

The particular foams described in the said prior art document do not contain any organic plasticiser and are dried to provide superabsorbency. They are generally formed by polymerising at least one suitable hydrophilic olefin monomer compound in an aqueous solution containing a surfactant and about 0.1 to about 10% by weight of a crosslinking agent having at least two alkenyl groups; introducing gas into the monomer solution during the polymerisation step to form the foamed polymer matrix; and drying the foam.

The Examples of the said prior art patent show the use of sodium bicarbonate as a carbon dioxide blowing agent to generate the gas, although the general description mentions also mechanical introduction of gas into the monomer solution. The introduction of gas into the monomer solution during the polymerisation step is inconvenient, and would generally limit the polymerisation procedure to small batchwise production.

The foams described in U.S. Pat. No. 5,750,585 swell slowly on contact with water, typically over a time period of about 1 to 3 hours (see the Figures in the prior art patent). This slowness of water uptake makes the foams unsuitable for use in the applications contemplated in the present invention. The relatively low density of the foam makes it unsuitable for forming into films and sheets having acceptable mechanical strength.

U.S. Pat. No. 6,136,873 (Hahnle et al), the disclosure of which is incorporated herein by reference, describes certain superabsorbent hydrogel foams. The preferred density of the foam is stated generally to be between 0.05 and 0.7 g/cm³.

The prior art foams are stated to have potential utility as superabsorbents in diapers, sanitary towels and incontinence articles, and in certain other conventional uses for superabsorbents. Dressing material for covering wounds is mentioned as one potential application (column 15, lines 24 to 26).

The prior art document contains extensive lists of possible monomers and monomer mixtures for use in the polymerisable mixture. However, all the examples use a mixture of acrylic acid and sodium acrylate.

The particular foams described in the said prior art document may contain certain plasticisers and are stated to be usually dried after polymerisation, preferably to a water content of between 15 to 35% by weight.

The gas introduced into the monomer mixture is stated to be “fine bubbles of a gas inert to free radicals”. Examples show the use of mechanical stirring under an atmosphere of argon or carbon dioxide.

The foams described in U.S. Pat. No. 6,136,873 swell on contact with water, the absorption speed being reported as the parameter AS in the Examples. As used therein, AS=20/t, where t=the time for a 1 g piece of the foam to absorb 20 g of water (i.e. a 2000% uptake). While the water uptake rate appears to be faster than the foams reported in U.S. Pat. No. 5,750,585, the manufacturing process is inconvenient in view of the need for an inert gas atmosphere, and is most suitable only for batchwise production.

A large amount of research has been conducted into unfoamed, relatively non-porous, hydrogels based on hydrophilic polymers, e.g. for use as skin adhesives for a range of applications in skin-adhesive articles. Such materials exhibit a range of properties which make them suitable for skin adhesives. Representative references include PCT Patent Applications Nos. WO-97/24149, WO-97/34947, WO-00/06214, WO-00/06215, WO-00/07638, WO-00/46319, WO-00/65143 and WO-01/96422, the disclosures of which are incorporated herein by reference.

### BRIEF DESCRIPTION OF THE INVENTION

The present invention is based on our surprising finding that porous hydrogels can be made in a convenient manner with very acceptable water uptake speeds. The manufacturing process, particularly at the polymerisation stage, can be batchwise, partially continuous or continuous. The porous hydrogels can be prepared in sheet or layer form. The porous hydrogels are characterised by an internal cellular structure. The porous hydrogels can combine the requirements of good mechanical strength and good fluid absorption capacity, optionally also with gel flexibility and skin tackiness.

The expressions “comonomer”, “monomer” and like expressions used herein include ionic and non-ionic monomers and monomer mixtures. The expressions “polymerise”, “polymers” and like expressions include both homopolymerisation and copolymerisation, and the products thereof.

According to a first aspect of the present invention, there is provided a hydrogel composition comprising a first portion which comprises a flexible plasticised hydrophilic polymer matrix having an internal cellular structure, and a second portion which comprises a flexible plasticised hydrophilic polymer matrix having relatively continuous internal structure.

The first portion may comprise a porous foam having an internal cellular structure such that the volume ratio of cell void to matrix is greater than about 1:3, more preferably greater than about 1:1, and the second portion may comprise a relatively non-porous matrix, which may have substantially no cell voids or only occasional cell voids (e.g. a volume ratio of cell void to matrix less than about 1:10, for example less than about 1:20). The said second portion of the hydrogel composition will be referred to herein as “continuous”, which expression is used in the relative sense explained above.

It is preferred that the said first, relatively porous, portion of the hydrogel composition has a first water uptake rate and the said second, relatively non-porous, portion of the hydrogel composition has a second water uptake rate which is less than the first. The first water uptake rate may be very fast, e.g. comparable with the rate of absorption of water by absorbent paper kitchen roll. The absorption capacity of the hydrogel composition will generally be at least about 30% by weight (i.e. the weight of water taken up and held at saturation will be at least about 30% of the weight of the hydrogel composition used), and may be as much as about 20000%. More typically, the absorption capacity of the hydrogel composition will be between about 300% and about 10000%. For convenience, the said first portion of the hydrogel composition will be referred to herein as “porous”, which expression is used in the relative sense explained above.

According to a second aspect of the present invention, there is provided a process for the preparation of a porous hydrogel, e.g. a hydrogel foam, which comprises polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles. When used to prepare the hydrogel composition according to the first aspect of the invention, the said first portion of the polymerisable mixture forms the porous portion of the hydrogel composition after polymerisation, and the said second portion of the polymerisable mixture forms the continuous portion of the hydrogel composition after polymerisation. The first portion of the polymerisable mixture preferably has a bubble to mixture volume ratio greater than about 1:3, more preferably greater than about 1:1, and the second portion of the polymerisable mixture preferably has substantially no bubbles or only occasional bubbles (e.g. a volume ratio of bubbles to mixture less than about 1:10, for example less than about 1:20).

The polymerisation step in the process according to the second aspect of the present invention is preferably a free radical polymerisation performed in air using a polymerisation inducing device such as a heat, light (e.g. UV light) or other radiation source which is in relative motion with respect to the polymerisable mixture. In this way, a moving line-wise polymerisation procedure can take place, rather than the static batchwise procedures available from the prior art. The polymerisable mixture is preferably laid down in sheet or layer form on a suitable support arrangement for the polymerisation procedure, whereby the first portion of the polymerisable mixture typically sits on the second portion in the manner of a “head” on beer.

Certain porous hydrogel compositions are novel per se, and they and the preferred process for their preparation constitute further aspects of the present invention.

According to a third aspect of the present invention, there is provided a porous hydrogel composition comprising a flexible plasticised hydrophilic polymer matrix having an internal cellular structure, wherein the hydrophilic polymer is selected from polymers of any of the following monomers:
2-acrylamido-2-methylpropane sulphonic acid or a substituted derivative or salt thereof;
acrylic acid (3-sulphopropyl) ester or a substituted derivative or salt thereof;
a non-ionic monomer containing an alkyl or alkylene or substituted alkyl or alkylene group linked to a carbon-carbon double bond via an amido or alkylamido function (e.g. diacetone acrylamide, a vinyl lactam, an N-alkylated acrylamide, an N,N-dialkylated acrylamide, N-vinyl pyrrolidone or N-acryloyl morpholine);
any mixture of any of the foregoing with each other or with one or more comonomer;
a monomer/comonomer pair consisting of a first monomer comprising one or more pendant anionic group and a second monomer comprising one or more pendant cationic group, preferably such a pair in which the relative amounts of the said monomers in the pair are such that the anionic groups and the cationic groups are present in essentially equimolar quantities;
any mixture of the said monomer/comonomer pair with any of the foregoing.

The porous hydrogel composition according to the third aspect of the present invention may comprise a porous foam having an internal cellular structure such that the volume ratio of cell void to matrix is greater than about 1:3, more preferably greater than about 1:1.

It is preferred that the said porous hydrogel composition has a very fast water uptake rate, e.g. comparable with the rate of absorption of water by absorbent paper kitchen roll. The absorption capacity of the hydrogel composition will generally be at least about 30% by weight (i.e. the weight of water taken up and held at saturation will be at least about 30% of the weight of the hydrogel composition used), and may be as much as about 20000%. More typically, the absorption capacity of the hydrogel composition will be between about 300% and about 10000%.

According to a fourth aspect of the present invention, there is provided a process for the preparation of a porous hydrogel composition according to the third aspect of the present invention, which comprises polymerising a polymerisable mixture comprising a hydrophilic monomer selected from monomers and monomer mixtures recited in the third aspect of the present invention, wherein the polymerisable mixture includes introduced gas bubbles.

Certain aspects of such a manufacturing process are more generally novel and inventive.

According to a fifth aspect of the present invention, there is provided a process for the preparation of a porous hydrogel composition, and porous hydrogel compositions prepared thereby, the process comprising polymerising a polymerisable mixture comprising a hydrophilic monomer and optionally one or more comonomer, wherein the polymerisable mixture includes bubbles consisting predominantly of air, the bubbles having been introduced into the mixture under an atmosphere consisting predominantly of air, and the mixture having been laid down for the said polymerisation after introduction of the bubbles into the polymerisable mixture but before polymerisation.

It is particularly preferred that the fourth and fifth aspects of the present invention are employed in combination.

The polymerisable mixture in the fourth and fifth aspects of the present invention preferably has a bubble to mixture volume ratio greater than about 1:3, more preferably greater than about 1:1.

The polymerisation step in the process according to the fourth and fifth aspects of the present invention is preferably a free radical polymerisation performed in air using a polymerisation inducing device such as a heat, light (e.g. UV light) or other radiation source which is in relative motion with respect to the polymerisable mixture. In this way, a moving line-wise polymerisation procedure can take place, rather than the static batchwise procedures available from the prior art. The polymerisable mixture is preferably laid down in sheet or layer form on a suitable support arrangement for the polymerisation procedure.

The procedures of laying down the gassed (foamed) polymerisable mixture preferably comprises casting the gassed mixture into the form of a relatively thin sheet, e.g. up to about 8 mm thick.

According to a sixth aspect of the present invention, there is provided a bioadhesive article adapted to be adhered to skin in use, the article comprising an adhesive for contacting the skin and a substrate supporting the adhesive, wherein the adhesive comprises a bioadhesive porous plasticised hydrophilic polymer having an internal cellular structure. The polymer may preferably be the hydrogel composition according to the first or third aspect of the present invention, or prepared according to the second, fourth or fifth aspect of the present invention, and is preferably in sheet or layer form. Where the hydrophilic polymer is a hydrogel composition in accordance with the first aspect of the present invention, the said continuous portion of the hydrogel composition will preferably form the skin-contacting surface of the adhesive. The skin-contacting portion of the hydrogel composition is preferably overlain with a protective flexible release layer prior to use of the article. At the time of use, the release layer is peeled away and may be discarded.

Most generally, a release layer is suitably applied to the hydrogel/adhesive polymer layer prior to the polymerisation procedure, e.g. by the release layer providing an upper surface of the support arrangement for the polymerisable mixture, onto which the polymerisable mixture is laid down.

The porous hydrophilic polymer or hydrogel composition used in this invention may be electrically conductive and constitute a skin-contacting adhesive portion of a biomedical electrode. Such a polymer will typically provide good current dispersion over the skin-electrode interface.

A bioadhesive wound or burn dressing typically comprises an absorbent member adapted to contact a wearer's skin in the location of a wound or burn, and a sheet backing member supporting the absorbent member, the sheet backing member including a portion which extends beyond the absorbent member and defines a skin-directed surface which carries a pressure-sensitive adhesive for securement of the dressing to the wearer's skin.

According to a seventh aspect of the present invention, there is provided a wound or burn dressing comprising an absorbent member adapted to contact a wearer's skin in the location of a wound or burn, and a sheet backing member supporting the absorbent member, the sheet backing member including a portion which extends beyond the absorbent member and defines a skin-directed surface which carries a pressure-sensitive adhesive for securement of the dressing to the wearer's skin, wherein the said absorbent member comprises a porous hydrophilic polymer having an internal cellular structure. The porous hydrophilic polymer may preferably be the hydrogel composition according to the first or third aspect of the present invention, or prepared according to the second, fourth or fifth aspect of the present invention, and is preferably in sheet or layer form. Where the hydrophilic polymer is a hydrogel composition in accordance with the first aspect of the present invention, the said continuous portion of the hydrogel composition will preferably form the skin-contacting surface of the absorbent member.

The sheet backing member is formed of any suitable material, e.g. a polymer (which may be foamed or unfoamed, or any combination thereof) such as polyurethane, or a fabric (which may comprise natural fibres, synthetic fibres or any combination thereof, and may be woven or non-woven). The sheet backing member may have any suitable structure, e.g. a web, film, sheet, net or any combination thereof

The pressure-sensitive adhesive is any suitable skin-compatible adhesive, e.g. an acrylic-based polymeric pressure-sensitive adhesive; a bioadhesive hydrogel or gel such as those described in the PCT Patent Applications mentioned above; or a bioadhesive porous plasticised hydrophilic polymer having an internal cellular structure, such as the hydrogel composition according to the first or third aspects of the present invention.

The skin-contacting adhesive parts of the dressing are preferably overlain with a protective flexible release layer prior to use of the dressing. At the time of use, the release layer is peeled away and may be discarded.

The porous hydrogel material in accordance with the present invention has a further utility deriving from its relatively rapid rate of absorption of liquids. This utility relates to the ability of the material to imbibe secondary components of a desired end product, which may be brought into contact, in liquid form, with the hydrogel material during the manufacturing process. Because of the speed of absorption achieved, such imbibing of secondary components can take place immediately or shortly after the polymerisation, preferably while the hydrogel is still on the same support arrangement as it was when polymerisation took place.

Such secondary hydrogel components may include, for example, liquid dispersions or solutions of conventional additives for hydrogels, such as electrolytes, pH regulators, colorants, chloride sources, bioactive compounds such as antimicrobials, antibiotics, antiseptics, haemostatic agents (such as calcium salts), wound healing agents, pharmaceuticals and drugs, burn healing agents, skin cooling agents, skin moisturizing agents, and skin warming agents, aroma agents, perfumes, fragrances and scents.

The secondary hydrogel component may also comprise polymer precursors in liquid form, such as dispersions or solutions of monomers or monomer mixtures in association with curing systems, or molten or dispersed or dissolved liquid forms of polymers or other (e.g. natural, synthetic or semi-synthetic) gel materials such as alginates (e.g. calcium alginate). When such secondary hydrogel components are added to the formed porous hydrogel of the present invention, e.g. immediately or shortly after polymerisation, the liquid is rapidly taken up into the cellular structure, where it may be dried, cured or set to create a secondary gel within the voids of the cellular structure. In the case of an open-cell structure of the hydrogel, the secondary gel may have relatively continuous domains within the structure, corresponding to the connectivity of the cellular structure. The secondary structure can be used in this way to increase the mechanical strength of the hydrogel material.

According to an eighth aspect of the present invention, there is provided a process for the preparation of a hydrogel composition, which comprises preparing a porous hydrogel composition in sheet or layer form by polymerising a pre-gel mixture on a suitable support arrangement with the upper face of the sheet or layer being porous, and applying to the porous upper face of the sheet or layer, while the sheet or layer is on the support arrangement on which the pre-gel mixture was polymerised, a liquid composition comprising a secondary component of the hydrogel composition or a precursor thereof, followed by setting, curing or drying of the secondary component within the porous structure if desired. It is preferred that the application of the liquid composition comprising the secondary component of the hydrogel composition or the precursor thereof will take place on the same day as the polymerisation to form the porous hydrogel material, most preferably within about three hours, e.g. up to about 90 minutes, after the polymerisation, and the any subsequent desired setting, curing or drying will take place on the same day as the application of the liquid composition comprising the secondary component of the hydrogel composition or the precursor thereof, most preferably within about three hours, e.g. up to about 90 minutes, after the said application.

The hydrogel material so formed can then be packed and sealed in conventional manner, or may be further processed, e.g. into a manufactured article comprising the hydrogel, in conventional manner or as described herein, before packing and sealing. By performing the post-imbibing procedure in situ immediately or soon after the polymerisation of the porous hydrogel, the manufacturing process can be considerably simplified, and the chances of bacterial infection or dirt contamination of the hydrogel material considerably reduced, in view of the increased potential for automation and the potential for reduction of human involvement and handling of the product.

According to a ninth aspect of the present invention, there is provided a porous hydrogel material having an internal cellular structure and containing within at least some of the cells one or more secondary hydrogel component selected from electrolytes, pH regulators, colorants, chloride sources, bioactive compounds such as antimicrobials, antibiotics, antiseptics, haemostatic agents (such as calcium salts), wound healing agents, pharmaceuticals and drugs, burn healing agents, skin cooling agents, skin moisturizing agents, and skin warming agents, aroma agents, perfumes, fragrances, scents, polymers, and other (e.g. natural, synthetic or semi-synthetic) gel materials such as alginates (e.g. calcium alginate).

The preferences for components, manufacturing methods and uses of the hydrogel materials described herein apply equally to hydrogel materials formed by the method of the eighth aspect of the present invention and the hydrogel materials according to the ninth aspect of the present invention.

In connection with the present invention, we have also found that a porous portion (layer) of the porous hydrogel material described herein, having an internal structure comprising a predominantly open-cell foam, can by suitable control of the manufacturing processes be made especially thin, for example less than about 0.7 mm in thickness, e.g. less than about 0.5 mm in thickness.

Surprisingly, we have found that when a porous layer of an ionic sheet hydrogel is made thin, and especially when the thin open-celled hydrogel material is underlain by an essentially non-porous layer, the swelling behaviour of the overall structure on imbibing of an external liquid is modified, and in particular the tendency to swell substantially in the direction orthogonal (normal) to the plane of the sheet (the so-called “z-direction”, this expression deriving from the conventional naming of the dimensions in a three-dimensional mathematical model) is significantly reduced. This creates a porous structure which is dimensionally constant even when imbibing significant volumes of external liquid. Such “z-restricted” swelling behaviour is advantageous in many applications, for example wound and burn care applications. As the thickness of the open-cell layer of the hydrogel increases, however, then the extent of swelling normal to the plane increases. The degree of swelling in the z-direction is preferably less than about 10% of the total thickness of the structure. The thickness of the underlying non-porous layer is preferably greater than 0.05 mm.

Preferably a 0.1 ml drop of normal saline applied to one point of the porous surface portion of such a hydrogel will after one minute spread to an area greater than 45 sq.mm, even more preferably greater than 70 sq.mm, and even more preferably to greater than 100 sq.mm.

The essentially non-porous layer underlying the thin porous layer may suitably be of the same hydrogel material as the porous layer, but may alternatively be of a different hydrogel material, of a non-hydrogel material, or any combination of any of these materials. The non-porous layer may be a mono-, bi- or multi-layer structure, and in the case of more than one layer the layers may be of the same or different materials in relation to each other and to the porous layer. The non-porous layer and component portions thereof may typically be continuous, closed-cell, predominantly closed-cell, or any combination thereof, or any other structure provided that the porosity to external liquids is substantially less than that of the thin porous layer.

Without being bound by theory, it seems that the difference in porosity between the porous and the non-porous portions causes fluid placed onto the porous part of a sheet of the hydrogel to spread laterally in the plane of the sheet (so-called “x,y-spread”) to a greater extent than porous hydrogels possessing a greater depth of open-cell structure at the surface. In effect, the porous layer can be considered to “wick” the applied fluid relatively rapidly away from the point of application, and for some reason not fully understood this effect seems to overtake any tendency of the hydrogel to swell. Even with only a very thin open-cell hydrogel layer, the capacity of an ionic hydrogel to imbibe and hold applied external water is enormous, so there is ordinarily no danger of saturation of the porous portion.

In accordance with a tenth aspect of the present invention, there is provided a water-absorbent structure comprising a porous hydrogel portion which comprises a flexible plasticised hydrophilic polymer matrix having a predominantly open-cell internal cellular structure, and a relatively non-porous further portion underlying the porous hydrogel portion, wherein the porous hydrogel portion comprises a sheet or layer of thickness less than about 0.7 mm.

The relatively non-porous further portion underlying the porous hydrogel portion may be of the same hydrogel material as the porous portion, but may alternatively be of a different hydrogel material, of a non-hydrogel material, or any combination of any of these materials. The underlying portion may be present as a layer. The underlying portion may be a mono-, bi- or multi-layer structure, and in the case of more than one layer the layers may be of the same or different materials in relation to each other and to the porous hydrogel portion. The underlying portion and component portions thereof may typically be continuous, closed-cell, predominantly closed-cell, or any combination thereof, or any other structure provided that the porosity to external liquids is substantially less than that of the thin porous hydrogel portion.

The water-absorbent structure with z-restricted swelling characteristics may be prepared by a number of methods. Where the structure consists of a hydrogel haying porous and non-porous portions, a process similar to that of the second aspect of the present invention may be used, but with additional control of the mixing of the ingredients of the polymerisable mixture or some of them, as described below. Where the structure consists of a hydrogel porous layer overlying a portion composed of a different hydrogel material or a non-hydrogel material, the portions may be prepared separately, the porous layer for example using the process of the fourth or fifth aspects of the present invention, and the structure assembled after polymerisation. Again, the formation of the porous layer may be subject to control of the mixing of the ingredients of the polymerisable mixture or some of them, as described below.

Examples of suitable non-porous non-hydrogel materials for use as potential non-porous portions of the water-absorbent structure according to the tenth aspect of the present invention will be well known to those of ordinary skill in this art. Suitable non-porous sheet materials bondable to hydrogels include non-porous synthetic polymer films and sheets, of which many suitable examples are commercially available.

In accordance with an eleventh aspect of the present invention, there is provided a process for the preparation of a hydrogel structure comprising a porous hydrogel portion which comprises a flexible plasticised hydrophilic polymer matrix having a predominantly open-cell internal cellular structure, and a relatively non-porous further portion underlying the porous portion, wherein the porous hydrogel portion is in the form of a sheet or layer of thickness less than about 0.7 mm, the process comprising forming by admixture of the ingredients a polymerisable mixture comprising one or more monomer, a curing system for the monomer(s), at least one surfactant and at least one plasticiser, the mixture including introduced gas bubbles, and polymerizing the polymerisable mixture, wherein during the forming of the polymerisable mixture at least some, preferably most or all, of the ingredients are mixed together using a rotary mixer, e.g. a propellor or paddle mixer, moving at a speed of more than about 500 rpm, more especially more than about 550 rpm, for example more than about 600 rpm, e.g. more than about 650 rpm, more than about 700 rpm, more than about 750 rpm, more than about 800 rpm, more than about 850 rpm, more than about 900 rpm, more than about 950 rpm or more than about 1000 rpm.

Generally speaking, it is found that the higher the speed of mixing the greater the level of closed-cell hydrogel relative to open-cell, i.e. the thinner the surface open-cell layer in the resultant structure. The absolute value of the mixing speed required will depend in part on the nature and amount of the surfactant used, and may need to be established through preliminary tests. Such tests will be well within the capacity of those of ordinary skill in this art.

The preferences for components, manufacturing methods and uses of the hydrogel materials described herein apply equally to hydrogel materials present in the structures according to the tenth aspect of the present invention and obtainable by the process according to the eleventh aspect of the present invention.

### DETAILED DESCRIPTION OF THE INVENTION

The Hydrogel Composition—Internal Structure

The internal cellular structure of the porous hydrogel composition or, when porous and continuous portions are present, the porous portion of the hydrogel composition, may be closed-cell throughout, open-cell throughout, or may have regions of closed-cell structure and regions of open-cell structure. Generally speaking, an open-cell structure will absorb fluid at a faster initial rate than a closed-cell structure, by reason of the interconnection of the internal cells.

Where porous and continuous portions of the hydrogel composition are present, they may suitably comprise layers, which may be of the same or different materials. The layers may be integrally formed or may be laminated together, optionally with intermediate bonding media

The said porous and continuous portions of such a hydrogel composition are preferably of the same material and integrally formed in a single polymerisation step.

In the polymerisation step, to be described in more detail below, a fluid pre-gel material is preferably gassed with bubbles of a gas, prior to laying down the pre-gel. The gas is preferably air. To prepare a hydrogel composition comprising porous and continuous portions, the lain down pre-gel is then preferably allowed or assisted to partially “drain”, by which is meant that a certain amount of the pre-gel material is allowed to revert to an essentially continuous, unfoamed, fluid state to form the second portion of the polymerisable mixture. By controlling the extent of draining, the relative thickness of the porous and continuous portions in the resulting cured hydrogel composition can be controlled. To prepare a porous hydrogel composition without a continuous portion, draining is avoided.

Where the porous and continuous portions of the hydrogel composition are present and are of different materials, the portions may suitably also be integrally formed in a single polymerisation step. We have found that the first (foam) portion of the laid down polymerisable mixture is usually relatively robust, and will not collapse if additional ingredients, e.g. comonomers, are added onto the mixture as a liquid dispersion, solution or mixture before the polymerisation step. In practice, the added ingredients percolate down through the first portion of the mixture and preferentially invade the fluid second portion below. By controlling the time allowed for this process, a range of differential-composition multi-layer porous hydrogels can be prepared conveniently, using a single polymerisation step to produce essentially the final hydrogel, without the need for lamination and handling of individual component layers after polymerisation or for laminar laying down of different polymerisable mixtures.

The Hydrogel Composition—External Form

The hydrogel composition may suitably be present in the form of a sheet having first and second major faces, each of said first and second major faces being in contact with a protective release layer, for example siliconised plastic or paper. Alternatively, the hydrogel composition may be present in the form of a sheet having first and second major faces, one of said first and second major faces being in contact with a protective release layer, for example siliconised plastic or paper, and the other of said first and second major faces being in contact with apart of a larger article, e.g. a backing member forming part of a wound or burn dressing, a biomedical electrode or another article. Particularly preferred are articles where a bioadhesive hydrogel layer is to be provided in use between the article and the skin of a patient. Such articles are exemplified below (see “Applications”). Still further, the hydrogel composition may be present in the form of a sheet having a woven or non-woven fabric, or a net, embedded therein.

The hydrogel sheets may typically have a thickness in the range of about 0.1 mm to about 10 mm, e.g. about 0.5 mm to about 10 mm. The thickness of the foam or film-foam structure can suitably be from about 0.1 mm to about 3 mm. When such sheets are in contact with a release sheet, for example a sheet of plastic or coated plastic (e.g. siliconised plastic) or paper or coated paper (e.g. siliconised paper), the hydrogel composition may suitably be coated at a surface weight of hydrogel in the range of about 0.5 kg/m² to about 2.5 kg/m².

For the preparation of a hydrogel composition in the form of a sheet, the process according to the invention may include initially forming a sheet of the pre-gel, and subsequently carrying out the polymerisation step so that the sheet hydrogel is formed in situ by the polymerisation reaction, as described in more detail below. In one embodiment, the resultant hydrogel may be used substantially as made, i.e. material is not substantially added to or removed from the resultant hydrogel composition, although in some cases some degree of subsequent conditioning and/or modification may be desirable, and in addition the post-processing of the eighth aspect of the present invention may advantageously be applied.

When the hydrogel composition contains water, the water may be present in any suitable amount. The typical range of water content is between 0 and about 95% by weight of the hydrogel. The hydrogel composition may conveniently be classified as “high water content” or “low water content”. The expression “high water content” refers particularly to hydrogel compositions comprising more than about 40% by weight of water, more particularly above about 50% by weight, and most preferably between about 60% and about 95% by weight. The expression “low water content” refers particularly to hydrogel compositions comprising up to about 40% by weight of water.

The Hydrogel Composition—Physical Parameters

The density of the hydrogel compositions of the present invention can be selected within a wide range, according to the materials used and the manufacturing conditions.

Generally speaking, the bulk density of the total hydrogel composition may be in the range of about 0.05 to about 1.5 g/cm³, more typically in the range of about 0.3 to about 0.8 g/cm³.

The water activity of the hydrogel compositions of the present invention typically lies within the range of 0 to about 0.96, as measured by an AquaLab Series 3TE water activity meter.

The water uptake rate of the hydrogel compositions of the present invention (or, where the composition includes a portion which is more porous than another portion, of the more porous portion) typically lies within the range of at least about 2 μl/s, more preferably between about 2 and about 1001 μl/s, as measured by the technique of applying a 5 μl drop of water from a syringe onto the face of the sheet hydrogel and measuring the reduction in volume of the drop over a period of 0.1 s starting from contact between the drop and the hydrogel, and extrapolating to a rate expressed as volume per second, the measurements being made using a Scientific and Medical Products DAT1100 dynamic contact angle analyser. A water uptake rate of, say, 25 μl/s, indicates complete absorption of the applied water in 0.2 s.

The water uptake rate of the hydrogel compositions of the first aspect of the present invention from the continuous portion side is typically less than the rate from the porous portion side, as measured by the same technique.

The absorption capacity of the hydrogel composition will generally be between about 30% and about 20000%. More typically, the absorption capacity of the hydrogel composition will be between about 300% and about 10000%.

Preparative Method—General

According to the invention, the processes for the preparation of porous hydrogels generally comprise polymerising a polymerisable mixture comprising at least one hydrophilic monomer, wherein the polymerisable mixture includes introduced gas bubbles, preferably, but not limited to, air bubbles.

In addition to the at least one hydrophilic monomer, a curing system should be present in the polymerisable mixture. The curing system typically includes at least one cross-linking agent and at least one suitable polymerisation initiator.

In one embodiment, the polymerisable mixture can comprise a first portion including a relatively high concentration of introduced gas bubbles and a second portion including a relatively low concentration of gas bubbles.

The polymerisation is preferably a free radical polymerisation of a fluid polymerisable mixture comprising
(1) a free radically polymerisable hydrophilic monomer, optionally together with at least one free radically polymerisable comonomer; and
(2) one or more cross-linking agent comprising a multifunctional unsaturated free radically polymerisable compound;
   the polymerisation being conducted in the presence or absence of a plasticiser, with the proviso that when the polymerisation is conducted in the absence of a plasticiser, a plasticiser is added to the polymer product of the polymerisation.

The polymerisable mixture (pre-gel) preferably includes the monomer(s) at a total monomer level of from about 5% to about 70% by weight of the total mixture, more particularly from about 10% to about 60% by weight, most preferably from about 15% to about 50% by weight.

When the polymerisation is conducted in the presence of a plasticiser, one or more different plasticiser and/or more of the same plasticiser may, if desired, be added to the polymer product of the polymerisation.

The plasticiser may be selected from aqueous and non-aqueous systems. Water or a mixture of water and a water-miscible organic plasticiser may suitably be used as an aqueous plasticiser. When a non-aqueous plasticiser is used, it may suitably be an organic plasticiser. Please see below (“Plasticiser”), for more details of plasticiser systems.

Preparation and Laying Down of the Polymerisable Mixture

In preparing hydrogel compositions in accordance with the invention, the ingredients are initially mixed to provide an ungassed polymerisable reaction mixture in the form of an initial fluid pre-gel.

The initial fluid pre-gel is then blown to introduce a gas into the mixture before polymerization. The gas can be introduced by mechanical means or by introduction of a blowing agent. Mechanical means include the use of a high speed blender or propeller under an atmosphere of the gas, or the introduction of the gas into the liquid through a capillary, nozzle or microporous surface. A blowing agent is any substance or combination of substances capable of producing the gas upon introduction into the mixture and application of any necessary initiation steps. Examples of blowing agents include carbonates or metal powders which react with acidic conditions to generate hydrogen or carbon dioxide, such as sodium bicarbonate, and chemical agents which liberate gas under the influence of heat such as dipotassium diazomethionate, N-nitroso-β-amino-ketones or sodium borohydride. Initiation of blowing will be achieved in any appropriate way, according to the chemicals being employed. Such initiation procedures will be well within the capacity of those skilled in the art.

The preferred gas for use in the present invention is air, which is preferably introduced into the initial pre-gel by mechanical means. To produce uniform cells in the porous portion of the hydrogel, the air bubbles introduced must be uniformly dispersed and the dispersion substantially maintained up until the point of gelation at polymerization.

The ingredients of the initial pre-gel are preferably mechanically mixed in such a way as to foam the mixture by the mechanical introduction of many small air bubbles. A typical mixing procedure would use a paddle stirrer for up to about 5 minutes at a paddle speed of up to about 800 rpm.

The viscosity of the initial pre-gel may need to be controlled. On the one hand, the viscosity should be low enough to permit effective introduction of the gas, as described below. On the other hand, the viscosity should not be so low that all the introduced gas bubbles rise to the surface and dissipate into the atmosphere before polymerization can take place to form the polymeric matrix. However, as explained above, a certain degree of “draining” is preferred, in order to obtain the hydrogel composition comprising integra 1 porous and continuous portions in one polymerization step. We have found that a viscosity of up to about 1000 mPas, more typically less than about 100 mPas, and most preferably lass than about 50 mPas (as measured in a Brookfield Viscometer with a S18 spindle in a closed volume at a speed of 20 rpm) is suitable for the initial pre-gel before introduction of gas, e.g. between about 10 and about 50 mPas.

The viscosity of the pre-gel mixture will rise as a result of this foaming procedure, to a typical range of between about 200 and about 1000 mPas (as measured in a Brookfield Viscometer with a S18 spindle in a closed volume at a speed of 2 rpm).

The gassed pre-gel mixture is then preferably laid down (cast) onto a suitable support arrangement prior to exposure to the source of the polymerising heat or radiation. The upper surface of the support arrangement is preferably provided by the sheet that will constitute the protective release layer to be provided with the hydrogel composition before use of any article in which it is included. Further details of a preferred embodiment of this release layer are given below (“Apparatus”).

In the time delay between casting onto the support arrangement and irradiation, the foamed pre-gel mixture may be allowed to “drain”, whereby a relatively bubble-free fluid layer forms under the foam layer, as previously described in connection with some aspects of the present invention.

The foam layer is usually mechanically stable enough that at least one further monomer or other desired component or components of the hydrogel composition can be added to the pre-gel mixture as it rests on the support arrangement awaiting polymerisation. Such additional components are typically applied on top of the foam layer in the form of a fluid dispersion, mixture or solution, e.g. in water, which then percolates down through the foam layer and mixes with any relatively bubble-free fluid layer underneath the foam. In this way, the composition of a continuous portion of the final hydrogel composition can be made different from that of the porous layer of the final composition, in a convenient way which still requires only one polymerisation step and can avoid or at least limit the degree of post-polymerisation handling, manufacture and processing of the product that is required.

A list of examples of suitable additional components is given below under the heading “Other Additives”.

The polymerisable mixture is then passed to the polymerisation step, which will now be discussed.

The Polymerisation Reaction

Any suitable free radical polymerisation reaction may be used, according to the monomers present in the pre-gel. The range of reactions and their appropriate initiation and other conditions will be well known to those of ordinary skill in this art.

For example, the free radical polymerisation may be initiated in generally known manner by light (photoinitiation), particularly ultraviolet light (UV photoinitiation); heat (thermal initiation); electron beam (e-beam initiation); ionising radiation, particularly gamma radiation (gamma initiation); non-ionising radiation, particularly microwave radiation (microwave initiation); or any combination thereof. The pre-gel mixture may include appropriate substances (initiators), at appropriate levels, e.g. up to about 5% by weight, more particularly between about 0.002% and about 2% by weight, which serve to assist the polymerisation and its initiation, in generally known manner.

In one embodiment, the process of the invention involves free radical polymerisation and the use of a photoinitiator or a combination of photo- and other initiation. Preferably the reaction mixture comprises an amount of photoinitiator of from about 0.003% to about 0.5%, and particularly from about 0.003% to about 0.4%, most particularly from about 0.009% to about 0.2%, by weight of the total polymerisation reaction mixture. If desired, the low levels of photoinitiator described in WO-01/96422 may be used.

In one preferred embodiment, the polymerisable mixture and the source of the polymerization initiator (e.g. the radiation source) move relative to one another for the polymerization step. In this way, a relatively large amount of polymerisable material can be polymerized in one procedure, more than could be handled in a static system. This moving system is referred to herein as continuous production, and is preferred.

Preferred photoinitiators include any of the following either alone or in combination:

Type I-α-hydroxy-ketones and benzilidimethyl-ketals e.g. Irgacure 651. These are believed on irradiation to form benzoyl radicals that initiate polymerisation. Photoinitiators of this type that are preferred are those that do not carry substituents in the para position of the aromatic ring. Examples include Irgacure 184 and Daracur 1173 as marketed by Ciba Chemicals, as well as combinations thereof.

A particularly preferred photoinitiator is 1-hydroxycyclohexyl phenyl ketone; for example, as marketed under the trade name Irgacure 184 by Ciba Speciality Chemicals. Also preferred are Daracur 1173 (2-hydroxy-2-propyl phenyl ketone) and mixtures of Irgacure 184 and Daracur 1173.

Photo-polymerisation is particularly suitable, and may be achieved using light, optionally together with other initiators, such as heat and/or ionizing radiation. Photoinitiation will usually be applied by subjecting the pre-gel reaction mixture containing an appropriate photoinitiation agent to ultraviolet (UV) light. The incident UV intensity, at a wavelength in the range from 240 to 420 nm, is typically greater than about 10 mW/cm². The processing will generally be carried out in a controlled manner involving a precise predetermined sequence of mixing and thermal treatment or history.

The UV irradiation time scale should ideally be less than 60 seconds, and preferably less than 10 seconds to form a gel with better than 95% conversion of the monomers. Those skilled in the art will appreciate that the extent of irradiation will be dependent on a number of factors, including the UV intensity, the type of UV source used, the photoinitiator quantum yield, the amount of monomer present, the nature of the monomer(s) present, the presence of dissolved oxygen, the presence of polymerisation inhibitor, the thickness of the reaction mixture when coated onto the substrate and the nature of substrate onto which the reaction mixture is coated.

After completion of the polymerisation reaction, and after any desired post-processing (such as provided by the eighth aspect of the present invention, described above), the hydrogel composition may typically be used immediately in a manufacturing procedure, e.g. to provide a skin-adhesive layer in an article, or a top release layer may be applied to the porous top side of the polymerised sheet material for storage and transportation of the porous hydrogel sheet.

Apparatus

The apparatus used is generally conventional and commercially available.

As mentioned above, however, according to one aspect of the present invention the support arrangement on which the gassed polymerisable mixture is laid down preferably supports, and thereby presents as its upper surface, the release layer.

In one preferred embodiment, the release layer is formed of a plastic sheet material, such as a polyolefin (e.g. polyethylene). The plastic material may optionally be coated with a non-stick material such as a silicone.

Ingredients of the Hydrogel Composition

The preferred hydrogel composition of the present invention comprises a plasticised three-dimensional matrix of cross-linked polymer molecules, and has sufficient structural integrity to be self-supporting even at very high levels of internal water content, with sufficient flexibility to conform to the surface contours of the human skin. Where the intended use of the hydrogel is in biomedical electrodes, wound dressings, and other applications where skin adhesion is desired, the hydrogel composition preferably has sufficient bioadhesion to adhere to the skin under all skin and moisture conditions likely to be encountered during use. Our PCT Patent Application No. WO-00/45864, the disclosure of which is incorporated herein by reference, describes a method whereby the skin adhesion performance of the hydrogel can be predicted and thereby tailored to particular applications.

The hydrogel compositions with which the present invention is concerned generally comprise, in addition to the cross-linked polymeric network, an aqueous plasticising medium and, where electrical conductivity is required, at least one electrolyte, whilst the materials and processing methods used are normally chosen to provide a suitable balance of adhesive and electrical properties for the desired application.

Ionic Monomers

The one or more ionic monomer, if present, will be water soluble and may be selected from: 2-acrylamido-2-methylpropane sulphonic acid or an analogue thereof or one of its salts (e.g. an ammonium or alkali metal salt such as a sodium, potassium or lithium salts); acrylic acid or an analogue thereof or one of its salts (e.g. an alkali metal salt such as a sodium, potassium or lithium salt); and/or a polymerisable sulphonate or a salt thereof (e.g. an alkali metal salt such as a sodium, potassium or lithium salt), more particularly acrylic acid (3-sulphopropyl) ester or an analogue thereof, or a salt thereof. The term “analogue” in this context refers particularly to substituted derivatives of 2-acrylamido-2-methylpropane sulphonic acid, of acrylic acid or of acrylic acid (3-sulphopropyl) ester.

A further category of ionic monomer that may be mentioned is a monomer/comonomer pair consisting of a first monomer comprising one or more pendant anionic group and a second monomer comprising one or more pendant cationic group, the relative amounts of the said monomers in the pair being such that the anionic groups and cationic groups are present in essentially equimolar quantities. The said anionic and cationic groups may be selected from groups which are salts of acid groups and groups which are salts of basic groups. The pendant groups in the first monomer are preferably the sodium, potassium, calcium, lithium and/or ammonium (individually or in any combination of one or more) salts of carboxylic acid, phosphoric acid and/or sulphonic acid. Sulphonic acid groups are most preferred. The pendant groups in the second monomer are preferably quaternary ammonium salts of halide (for example chloride), sulphate and/or hydroxide. Chloride and sulphate are most preferred.

A particularly preferred ionic monomer is a sodium salt of 2-acrylamido-2-methylpropane sulphonic acid, commonly known as NaAMPS, which is available commercially at present from Lubrizol as either a 50% aqueous solution (reference code LZ2405) or a 58% aqueous solution (reference code LZ2405A) and/or acrylic acid (3-sulphopropyl) ester potassium salt, commonly known as SPA or SPAK. SPA or SPAK is available commercially in the form of a pure solid from Raschig. In the case of polymers formable from a monomer/comonomer pair consisting of a first monomer comprising one or more pendant anionic group and a second monomer comprising one or more pendant cationic group, the relative amounts of the said monomers in the pair being such that the anionic groups and cationic groups are present in essentially equimolar quantities, these ionic monomers will provide suitable monomers comprising one or more pendant anionic group. In that case, suitable monomers comprising one or more pendant cationic group may, for example, be alkyl ester derivatives of acrylic acid in which the alkyl group carries a quaternised ammonium ion substituent, the counter-anion suitably being halide (for example chloride), sulphate and/or hydroxide. Acryloyloxyethyltrimethylammonium salts (e.g. the chloride) are particularly mentioned.

Non-Ionic Monomers

The one or more non-ionic monomer, if present, may preferably be water soluble and be selected from acrylamide or a mono- or di-N-alkylacrylamide or an analogue thereof. The term “analogue” in this in this context refers to non-ionic water soluble monomers containing an alkyl or substituted alkyl group linked to a carbon-carbon double bond via an amido or alkylamido (—CO.NH— or —CO.NR—) function. Examples of such analogues include diacetone acrylamide (N-1,1-dimethyl-3-oxobutyl-acrylamide), vinyl lactams, N-alkylated acrylamides, N,N-dialkylated acrylamides, N-vinyl pyrrolidone, N-acryloyl morpholine and any mixture thereof. N-acryloyl morpholine is particularly preferred.

Cross-Linking Agents

Conventional cross-linking agents are suitably used to provide the necessary mechanical stability and to control the adhesive properties of the hydrogel. The amount of cross-linking agent required will be readily apparent to those skilled in the art such as from about 0.01% to about 0.5%, particularly from about 0.05% to about 0.4%, most particularly from about 0.08% to about 0.3%, by weight of the total polymerisation reaction mixture. Typical cross-linkers include tripropylene glycol diacrylate, ethylene glycol dimethacrylate, triacrylate, polyethylene glycol diacrylate (polyethylene glycol (PEG) molecular weight between about 100 and about 4000, for example PEG400 or PEG600), and methylene bis acrylamide.

Organic Plasticisers

The one or more organic plasticiser, when present, may suitably comprise any of the following either alone or in combination: at least one polyhydric alcohol (such as glycerol, polyethylene glycol, or sorbitol), at least one ester derived therefrom, at least one polymeric alcohol (such as polyethylene oxide) and/or at least one mono- or poly-alkylated derivative of a polyhydric or polymeric alcohol (such as alkylated polyethylene glycol). Glycerol is the preferred plasticiser. An alternative preferred plasticiser is the ester derived from boric acid and glycerol. When present, the organic plasticiser may comprise up to about 45% by weight of the hydrogel composition.

Surfactants

Any compatible surfactant may optionally be used as an additional ingredient of the hydrogel composition. Surfactants can lower the surface tension of the mixture before polymerisation and thus aid processing. Non-ionic, anionic and cationic surfactants are preferred. The surfactant ideally comprises any of the surfactants listed below either alone or in combination with each other and/or with other surfactants. The total amount of surfactant, if present, is suitably up to about 10% by weight of the hydrogel composition, preferably from about 0.05% to about 4% by weight.

1. Non-Ionic Surfactants

Suitable non-ionic surfactants include, but are not limited to, those selected from the group consisting of the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles and most preferably about 5 to about 20 moles of ethylene oxide. Examples of such non-ionic ethoxylated fatty alcohol surfactants are the Tergitol™ 15-S series from Union Carbide and Brij™ surfactants from ICI. Tergitol™ 15-S surfactants include C₁₁-C₁₅ secondary alcohol polyethyleneglycol ethers. Brij™ 58 surfactant is polyoxyethylene(20) cetyl ether, and Brij™ 76 surfactant is polyoxyethylene(10) stearyl ether.

Other suitable non-ionic surfactants include, but are not limited to, those selected from the group consisting of the polyethylene oxide condensates of one mole of alkyl phenol containing from about 6 to 1.2 carbon atoms in a straight or branched chain configuration, with about 3 to about 100 moles of ethylene oxide. Examples of non-ionic surfactants are the Igepal™ CO and CA series from Rhone-Poulenc. Igepal™ CO surfactants include nonylphenoxy poly(ethyleneoxy) ethanols. Igepal™ CA surfactants include octylphenoxy poly(ethyloneoxy) ethanols.

Another group of usable non-ionic surfactants include, but are not limited to, those selected from the group consisting of block copolymers of ethylene oxide and propylene oxide or butylene oxide. Examples of such non-ionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers. The balance of hydrophobic and hydrophilic components within the surfactant together with the molecular weight are found to be important. Suitable examples are Pluronic L68 and Tetronic 1907. Particularly suitable examples are Pluronic L64 and Tetronic 1107.

Still other satisfactory non-ionic surfactants include, but are not limited to, those selected from the group consisting of sorbitan fatty acid esters, polyoxyethylene sorbitan fatty acid esters and polyoxyethylene stearates. Examples of such fatty acid ester non-ionic surfactants are the Span™, Tween™, and Myrj™ surfactants from ICI. Span™ surfactants include C₁₂-C₁₈ sorbitan monoesters. Tween™ surfactants include poly(ethylene oxide) C₁₂-C₁₈ sorbitan monoesters. Myrj™ surfactants include poly(ethylene oxide) stearates.

2. Anionic Surfactants

Anionic surfactants normally include a hydrophobic moiety selected from the group consisting of (about C₆ to about C₂₀) alkyl, alkylaryl, and alkenyl groups and an anionic group selected from the group consisting of sulfate, sulfonate, phosphate, polyoxyethylene sulfate, polyoxyethylene sulfonate, polyoxyethylene phosphate and the alkali metal salts, ammonium salts, and tertiary amino salts of such anionic groups.

Anionic surfactants which can be used in the present invention include, but are not limited to those selected from the group consisting of (about C₆ to about C₂₀) alkyl or alkylaryl sulfates or sulfonates such as sodium lauryl sulfate (commercially available as Polystep™ B-3 from Srepan Co.) and sodium dodecyl benzene sulfonate, (commercially available as Siponate™ DS-10 from Rhone-Poulenc); polyoxyethylene (about C₆ to about C₂₀) alkyl or alkylphenol ether sulfates with the ethylene oxide repeating unit in the surfactant below about 30 units, preferably below about 20 units, most preferably below about 15 units, such as Polystep™ B-1 commercially available from Stepan Co. and Alipal™ EP110 and 115 from Rhone-Poulenc; (about C₆ to about C₂₀) alkyl or alkylphenoxypoly (ethyleneoxy)ethyl mono-esters and di-esters of phosphoric acid and its salts, with the ethylene oxide repeating unit in the surfactant below about 30 units, preferably below about 20 units, most preferably below about 15 units, such as Gafac™ RE-510 and Gafac™ RE-610 from GAF.

3. Cationic Surfactants

Cationic surfactants useful in the present invention include, but are not limited to, those selected from the group consisting of quaternary ammonium salts in which at least one higher molecular weight group and two or three lower molecular weight groups are linked to a common nitrogen atom to produce a cation, and wherein the electrically-balancing anion is selected from the group consisting of a halide (bromide, chloride, etc.), acetate, nitrite, and lower alkosulfate (methosulfate etc.). The higher molecular weight substituent(s) on the nitrogen is/are often (a) higher alkyl group(s), containing about 10 to about 20 carbon atoms, and the lower molecular weight substituents may be lower alkyl of about 1 to about 4 carbon atoms, such as methyl or ethyl, which may be substituted, as with hydroxy, in some instances. One or more of the substituents may include an aryl moiety or may be replaced by an aryl, such as benzyl or phenyl.

In a preferred embodiment of the invention the surfactant comprises at least one propylene oxide/ethylene oxide block copolymer, for example such as that supplied by BASF Plc under the trade name Pluronic P65 or L64 or F68.

Other Additives

The hydrogel composition of the present invention may include one or more additional ingredients, which may be added to the pre-polymerisation mixture or the polymerised product, at the choice of the skilled worker. Such additional ingredients are selected from additives known in the art, including, for example, water, organic plasticisers, surfactants, polymers, electrolytes, pH regulators, colorants, chloride sources, bioactive compounds, personal and body care agents, and mixtures thereof. The polymers can be natural polymers (e.g. xanthan gum), synthetic polymers (e.g. polyoxypropylene-polyoxyethylene block copolymer or poly-(methyl vinyl ether alt maleic anhydride)), or any combination thereof. By “bioactive compounds” we mean any compound or mixture included within the hydrogel for some effect it has on living systems as opposed to the hydrogel, whether the living system be bacteria or other microorganisms or higher animals such as the intended user of articles incorporating the hydrogel. A biocidal biaoactive compound that may particularly be mentioned is citric acid.

Additional polymer(s), typically rheology modifying polymer(s), may be incorporated into the polymerisation reaction mixture at levels typically up to about 10% by weight of total polymerisation reaction mixture, e.g. from about 0.2% to about 10% by weight. Such polymer(s) may include polyacrylamide, poly-NaAMPS, polyethylene glycol (PEG), polyvinylpyrrolidone (PVP) or carboxymethyl cellulose.

A particularly preferred application is in the field of biomedical skin electrodes. When the hydrogels are intended for use in conjunction with Ag/AgCl medical electrodes, chloride ions are required to be present in order for the electrode to function. Potassium chloride and sodium chloride are commonly used. However any compound capable of donating chloride ions to the system may be used, for example, lithium chloride, calcium chloride, magnesium chloride or ammonium chloride. The amount that should be added is dependent on the electrical properties required and is typically about 0.5-8% by weight.

In general, an electrolyte (e.g. a salt such as a chloride as mentioned above or another salt such as a nitrate, for example sodium or calcium nitrate) will need to be included in the polymerisation reaction mixture in appropriate amounts, when the process is used to manufacture a hydrogel composition for use in an electrode.

The compositions prepared according to the present invention are used in biomedical electrodes in generally conventional manner, as will be readily understood by those skilled in this art. Such biomedical electrodes may include electrodes (suitably in patch form) for diagnostic, stimulation, therapeutic and electrosurgical use. The hydrogel compositions according to the present invention will typically provide good current dispersion over the skin-electrode interface, leading to potential benefits through reduction of electrical hot-spots.

Additional functional ingredients may also incorporated in the reaction mixture used in the invention, including bioactive compounds such as antimicrobial agents (e.g. citric acid, stannous chloride), enzymes, compounds providing a heating or cooling sensation to a patient's body, dermatologically active compounds and, for drug delivery applications, pharmaceutically active agents, the latter being designed to be delivered either passively (e.g. transdermally) or actively (e.g. iontophoretically) through the skin.

For use in pharmaceutical delivery devices for the delivery of pharmaceuticals or other active agents to or through mammalian skin, the compositions may optionally contain topical, transdermal or iontophoretic agents and excipients. The compositions may contain penetration-enhancing agents to assist the delivery of water or active agents into the skin. Non-limiting examples of penetration-enhancing agents for use in such applications include methyl oleic acid, isopropyl myristate, Azone™, Transcutol™ and N-methylpyrrolidone.

The additional ingredient may comprise an antimicrobial agent stable against light and radiation, comprising an effective amount of antimicrobial metal (e.g. silver) ions and stabilizing halide (e.g. chloride) ions, wherein the halide is present in an excess (preferably in a substantial molar excess such as around 500-fold excess) with respect to the amount of metal ions.

The hydrogel composition of the present invention preferably consists essentially of a cross-linked hydrophilic polymer of a hydrophilic monomer and optionally one or more comonomer, together with water and/or one or more organic plasticiser, and optionally together with one or more additives selected from surfactants, polymers, pH regulators, electrolytes, chloride sources, bioactive compounds and mixtures thereof, with less than about 10% by weight of other additives.

Applications

The hydrogel compositions described herein may suitably be used in a range of skin contact or covering applications where the composition is brought into contact either with skin or with an intermediary member which interfaces between the composition and the skin. The composition may be unsupported or may be supported on a part of a larger article for some specific use, e.g. a backing structure. The compositions may suitably be in the form of sheets, coatings, membranes, composites or laminates. Applications include patches, tapes, bandages, devices and dressings of general utility or for specific uses, including without limitation biomedical, skin care, personal and body care, palliative and veterinary uses such as, for example, skin electrodes for diagnostic (e.g. ECG), stimulation (e.g. TENS), therapeutic (e.g. defibrillation) or electrosurgical (e.g. electrocauterisation) use; dressings and reservoirs for assisting wound and burn healing, wound and burn management, skin cooling, skin moisturizing, skin warming, aroma release or delivery, decongestant release or delivery, pharmaceutical and drug release or delivery, perfume release or delivery, fragrance release or delivery, scent release or delivery, and other skin contacting devices such as absorbent pads or patches for absorbing body fluids (e.g. lactation pads for nursing mothers), hairpiece adhesives and clothing adhesives; and adhesive flanges and tabs for fecal collection receptacles, ostomy devices and other incontinence devices.

### EXAMPLES

The invention will be further described with reference to the following Examples, which should not be understood to limit the scope of the invention.

Test Methods

Pre-foam viscosity was determined using a Brookfield Viscometer with a S18 spindle in a closed volume at a speed of 20 rpm. The pre-cured foam viscosities were also determined using a Brookfield Viscometer with a S18 spindle in a closed volume at a speed of 2 rpm.

The rate of absorption of water on the continous layer and on the porous layer were determined by placing a 5 μl drop from a syringe and monitoring the drop volume on the surface of the material over the first 0.1 s. This was done using a Scientific and Medical Products DAT1100 dynamic contact angle analyser.

The rheology of the hydrogel foam composite was determined with a Rheometrics SR5 rheometer over a range from 0.1 to 100 rad/s.

Water activities of the foamed hydrogels were determined with an AquaLab Series 3TE water activity meter.

Preparative Methods and Compositions

### Examples 1 to 15

### Preparative Method and Apparatus

The method for making 200 g of hydrogel foam will be described below. It will be appreciated by those skilled in the art that this may be scaled up to enable semi-continuous or continuous hydrogel foam to be made.

200 g of hydrogel pre-foam formulation mix is added to a 500 ml vessel. A paddle stirrer is placed into the pre-foam formulation mix. The paddle is connected to an IKA RW 16 Basic mixer. The mix is stirred for three minutes at a speed of 500 to 600 rpm until the mix is frothy and has increased in viscosity. It will be appreciated that different mixing times and speeds may be employed depending on the extent of foaming required. At the end of the foaming period the paddle is removed from the vessel. The foam is then poured (cast) onto a suitable substrate surface (e.g. a film, embossed film, non woven or net substrate, made from natural or synthetic materials or combinations of both) and irradiated with UV light (for example from a medium pressure mercury arc lamp) to cure the foam. The resulting material is according to this invention a composite structure comprising a continuous hydrogel layer (as defined above) in contact with the substrate and a porous layer adjacent to it. By casting the foamed mix onto a moving substrate, a continuous roll of composite material can be produced at speeds from 0.5 m/min to 30 m/min. Variation of the extent of foaming and the time between casting the foam and then curing allows the thickness ratio of the continuous and porous layer portions of the hydrogel sheet to be altered and controlled.

### Examples 1 to 15

### Compositions

The compositions of the hydrogels prepared are shown below:

| | | Example | |
|---|---|---|---|
| | | Number | |
| | | 1 | 2 |
| N-Acryloylmorpholine | % | 0.0 | 0.0 |
| Sodium 2-acrylamido-2-methylpropane sulphonate | % | 31.3 | 56.8 |
| N,N-Dimethylaminoethylacrylate, methyl chloride quarternary salt | % | 26.2 | 0.0 |
| N,N-Dimethylamide | % | 0.0 | 0.0 |
| 3-Sulphopropyl acrylate potassium salt | % | 0.0 | 0.0 |
| Acrylic Acid | % | 0.0 | 0.0 |
| Sodium Acrylate | % | 0.0 | 0.0 |
| Glycerol | % | 9.9 | 0.0 |
| Water | % | 29.6 | 41.2 |
| Citric Acid | % | 0.0 | 0.0 |
| Silver Nitrate | % | 0.0 | 0.0 |
| Magnesium Chloride hexahydrate | % | 0.0 | 0.0 |
| Polyoxypropylene-Polyoxyethylene block co-polymer | % | 3.0 | 2.0 |
| Daracure 1173/Irgacure 280 15/20 | g/100 g | 0.0 | 0.0 |
| Daracure 1173/Irgacure 280 8/20 | g/100 g | 0.0 | 0.0 |
| Daracure 1173/Irgacure 280 6/20 | g/100 g | 0.0 | 0.0 |
| Daracure 1173/Irgacure 280 4/20 | g/100 g | 0.7 | 0.0 |
| Daracure 1173/Irgacure 280 1/20 | g/100 g | 0.0 | 0.6 |
| | | Example | |
| | | Number | |
| | | 3 | 4 |
| N-Acryloylmorpholine | % | 48.4 | 48.0 |
| Sodium 2-acrylamido-2-methylpropane sulphonate | % | 1.9 | 1.9 |
| N,N-Dimethylaminoethylacrylate, methyl chloride quarternary salt | % | 0.0 | 0.0 |
| N,N-Dimethylamide | % | 0.0 | 0.0 |
| 3-Sulphopropyl acrylate potassium salt | % | 0.0 | 0.0 |
| Acrylic Acid | % | 0.0 | 0.0 |
| Sodium Acrylate | % | 0.0 | 0.0 |
| Glycerol | % | 32.3 | 32.0 |
| Water | % | 14.3 | 14.1 |
| Citric Acid | % | 0.0 | 0.8 |
| Silver Nitrate | % | 0.0 | 0.0 |
| Magnesium Chloride hexahydrate | % | 0.0 | 0.0 |
| Polyoxypropylene-Polyoxyethylene block co-polymer | % | 3.2 | 3.2 |
| Daracure 1173/Irgacure 280 15/20 | g/100 g | 0.0 | 0.0 |
| Daracure 1173/Irgacure 280 8/20 | g/100 g | 0.0 | 0.0 |
| Daracure 1173/Irgacure 280 6/20 | g/100 g | 1.2 | 1.2 |
| Daracure 1173/Irgacure 280 4/20 | g/100 g | 0.0 | 0.0 |
| Daracure 1173/Irgacure 280 1/20 | g/100 g | 0.0 | 0.0 |
| | | Example | |
| | | Number | |
| | | 5 | 6 |
| N-Acryloylmorpholine | % | 28.4 | 48.7 |
| Sodium 2-acrylamido-2-methylpropane sulphonate | % | 0 | 5.7 |
| N,N-Dimethylaminoethylacrylate, methyl chloride quarternary salt | % | 0 | 0 |
| N,N-Dimethylamide | % | 0.0 | 0 |
| 3-Sulphopropyl acrylate potassium salt | % | 0.0 | 0 |
| Acrylic Acid | % | 0.0 | 0 |
| Sodium Acrylate | % | 0.0 | 0 |
| Glycerol | % | 14.3 | 39 |
| Water | % | 18.9 | 4.1 |
| Citric Acid | % | 0 | 0 |
| Silver Nitrate | % | 0.0 | 0 |
| Magnesium Chloride hexahydrate | % | 36 | 0 |
| Polyoxypropylene-Polyoxyethylene block co-polymer | % | 2.4 | 2.4 |
| Daracure 1173/Irgacure 280 15/20 | g/100 g | 0.0 | 0.4 |
| Daracure 1173/Irgacure 280 8/20 | g/100 g | 0.1 | 0.0 |
| Daracure 1173/Irgacure 280 6/20 | g/100 g | 0.0 | 0.0 |
| Daracure 1173/Irgacure 280 4/20 | g/100 g | 0.0 | 0.0 |
| Daracure 1173/Irgacure 280 1/20 | g/100 g | 0.0 | 0.0 |
| | | Example | |
| | | Number | |
| | | 7 | 8 |
| N-Acryloylmorpholine | % | 0.0 | 0.0 |
| Sodium 2-acrylamido-2-methylpropane sulphonate | % | 7.6 | 0.0 |
| N,N-Dimethylaminoethylacrylate, methyl chloride quarternary salt | % | 0.0 | 0.0 |
| N,N-Dimethylamide | % | 0.0 | 0.0 |
| 3-Sulphoproply acrylate potassium salt | % | 0.0 | 0.0 |
| Acrylic Acid | % | 0.0 | 0.0 |
| Sodium Acrylate | % | 25.1 | 28.5 |
| Glycerol | % | 0.0 | 0.0 |
| Water | % | 64.1 | 66.8 |
| Citric Acid | % | 0.0 | 0.0 |
| Silver Nitrate | % | 0.0 | 0.0 |
| Magnesium Chloride hexahydrate | % | 0.0 | 0.0 |
| Polyoxypropylene-Polyoxyethylene block co-polymer | % | 3.3 | 0.0 |
| Daracure 1173/Irgacure 280 15/20 | g/100 g | 0.0 | 0.0 |
| Daracure 1173/Irgacure 280 8/20 | g/100 g | 0.0 | 0.0 |
| Daracure 1173/Irgacure 280 6/20 | g/100 g | 0.0 | 0.0 |
| Daracure 1173/Irgacure 280 4/20 | g/100 g | 0.0 | 0.0 |
| Daracure 1173/Irgacure 280 1/20 | g/100 g | 0.8 | 0.7 |
| | | Example | |
| | | Number | |
| | | 9 | 10 |
| N-Acryloylmorpholine | % | 0.00 | 0.0 |
| Sodium 2-acrylamido-2-methylpropane sulphonate | % | 56.77 | 32.8 |
| N,N-Dimethylaminoethylacrylate, methyl chloride quarternary salt | % | 0.00 | 0.0 |
| N,N-Dimethylamide | % | 0.00 | 0.0 |
| 3-Sulphopropyl acrylate potassium salt | % | 0.00 | 9.6 |
| Acrylic Acid | % | 0.00 | 1.9 |
| Sodium Acrylate | % | 0.00 | 0.0 |
| Glycerol | % | 0.00 | 33.7 |
| Water | % | 41.11 | 23.0 |
| Citric Acid | % | 0.00 | 0.0 |
| Silver Nitrate | % | 0.01 | 0.0 |
| Magnesium Chloride hexahydrate | % | 0.00 | 0.0 |
| Polyoxypropylene-Polyoxyethylene block co-polymer | % | 2.11 | 1.9 |
| Daracure 1173/Irgacure 280 15/20 | g/100 g | 0.00 | 0.0 |
| Daracure 1173/Irgacure 280 8/20 | g/100 g | 0.00 | 0.0 |
| Daracure 1173/Irgacure 280 6/20 | g/100 g | 0.00 | 0.0 |
| Daracure 1173/Irgacure 280 4/20 | g/100 g | 0.00 | 0.0 |
| Daracure 1173/Irgacure 280 1/20 | g/100 g | 0.7 | 0.1 |
| | | Example | |
| | | Number | |
| | | 11 | 112 |
| N-Acryloylmorpholine | % | 0.0 | 0.0 |
| Sodium 2-acrylamido-2-methylpropane sulphonate | % | 0 | 0 |
| N,N-Dimethylaminoethylacrylate, methyl chloride quarternary salt | % | 0.0 | 0.0 |
| N,N-Dimethylamide | % | 47.5 | 0.0 |
| 3-Sulphopropyl acrylate potassium salt | % | 0.0 | 49.0 |
| Acrylic Acid | % | 0.0 | 0.0 |
| Sodium Acrylate | % | 0.0 | 0.0 |
| Glycerol | % | 40.0 | 24.2 |
| Water | % | 10.0 | 24.3 |
| Citric Acid | % | 0.0 | 0.0 |
| Silver Nitrate | % | 0.0 | 0.0 |
| Magnesium Chloride hexahydrate | % | 0.0 | 0.0 |
| Polyoxypropylene-Polyoxyethylene block co-polymer | % | 2.5 | 2.5 |
| Daracure 1173/Irgacure 280 15/20 | g/100 g | 0.0 | 0.0 |
| Daracure 1173/Irgacure 280 8/20 | g/100 g | 0.0 | 0.0 |
| Daracure 1173/Irgacure 280 6/20 | g/100 g | 0.7 | 0.0 |
| Daracure 1173/Irgacure 280 4/20 | g/100 g | 0.0 | 0.3 |
| Daracure 1173/Irgacure 280 1/20 | g/100 g | 0.0 | 0.0 |

| | | Example |
|---|---|---|
| | | Number |
| | | 13 |
| N-Acryloylmorpholine | % | 0.0 |
| Sodium 2-acrylamido-2-methylpropane sulphonate | % | 0 |
| N,N-Dimethylaminoethylacrylate, methyl chloride quarternary salt | % | 28.2 |
| NN Dimethylamide | % | 0.0 |
| 3-Sulphonyl acrylate potassium salt | % | 0 |
| Acrylic Acid | % | 0.0 |
| Sodium Acrylate | % | 0.0 |
| Glycerol | % | 47.3 |
| Water | % | 18.9 |
| Citric Acid | % | 0.0 |
| Silver Nitrate | % | 0.0 |
| Magnesium Chloride hexahydrate | % | 0.0 |
| Polyoxypropylene-Polyoxyethylene block co-polymer | % | 5.5 |
| Daracure 1173/Irgacure 280 15/20 | g/100 g | 0.0 |
| Daracure 1173/Irgacure 280 8/20 | g/100 g | 0.9 |
| Daracure 1173/Irgacure 280 6/20 | g/100 g | 0.0 |
| Daracure 1173/Irgacure 280 4/20 | g/100 g | 0.0 |
| Daracure 1173/Irgacure 280 1/20 | g/100 g | 0.0 |

Compositions containing thickeners and or fillers

| | | Example | |
|---|---|---|---|
| | | Number | |
| | | 14 | 15 |
| N-Acryloylmorpholine | % | 0.0 | 0.0 |
| Sodium 2-acrylamido-2-methylpropane sulphonate | % | 31.3 | 34.6 |
| N,N-Dimethylaminoethylacrylate, methyl chloride quarternary salt | % | 26.2 | 28.9 |
| Glycerol | % | 0.0 | 0.0 |
| Water | % | 38.5 | 32.7 |
| Poly (methyl vinyl ether alt maleic anhydride) | % | 1.0 | 0.0 |
| Xanthan gum | % | 0.0 | 0.5 |
| Polyoxypropylene-Polyoxyethylene block co-polymer | % | 3.0 | 3.3 |
| Daracure 1173/Irgacure 280 15/20 | g/100 g | 0.0 | 0.0 |
| Daracure 1173/Irgacure 280 8/20 | g/100 g | 0.0 | 0.0 |
| Daracure 1173/Irgacure 280 6/20 | g/100 g | 0.7 | 0.7 |
| Daracure 1173/Irgacure 280 4/20 | g/100 g | 0.0 | 0.0 |
| Daracure 1173/Irgacure 280 1/20 | g/100 g | 0.0 | 0.0 |

### Examples 16 to 49

### Preparative Method and Apparatus

The appropriate weight of N-acryloylmorpholine (ACMO) was added to the appropriate weight of water (Examples 16 to 37, 39, 40, 45, 46 to 49) or to the aqueous saturated or supersaturated liquid formed by gentle warming of a hydrated salt (see further details below) to about 60° C. (Examples 38, 41 to 44, 47 and 48). The surfactant Pluronic 65 (“P65”) was added to each aqueous composition thereby obtained.

For Examples 33, 34, 41 and 43, acrylic acid (AA) comonomer was also added with the ACMO. For Example 24, 2-acrylamido-2-methylpropane sulphonic acid sodium salt (NaAMPS) was also added with the ACMO (see discussion below). For Examples 39, 40, 45 and 47 to 49, a salt (see further details below) was also added, if necessary with gentle warming. For Examples 38 to 49, the salt was selected from calcium chloride hexahydrate (Examples 38 to 43), calcium nitrate tetrahydrate (Examples 44 and 45), a 50:50 weight mixture of calcium chloride hexahydrate and calcium nitrate tetrahydrate (Example 46), sodium chloride (Example 47) and magnesium chloride hexahydrate (Examples 48 and 49). The amounts of the AA and the salt are indicated in the table below. The appropriate weight of glycerol was added (Examples 20 to 30, 33 to 37, 42 and 43 only) and the mixture stirred for about 30 minutes. Amounts of these initial ingredients for Examples 16 to 37 are shown in parts by weight (normally out of 100, but out of 104 in the case of Example 24); amounts for Examples 38 to 49 are shown in grams.

A mixture of crosslinker (“XL”) and photoinitiator (“PI”) was made by adding the appropriate weight of IRR280 (PEG400 diacrylate, UCB Chemicals) (“280”) to the appropriate weight of photoinitiator, Daracur 1173 (Ciba Specialty Chemicals) (“1173”)”. The appropriate amount of this liquid mixture was added to the mixture, which was stirred for 1 hour, covered to exclude light. The figures for Examples 16 to 24, 27 and 31 to 35 in the table below show the percentage by weight of the initial mixture, at which the PI/XL mixture (6 parts by weight PI: 20 parts by weight XL) is added. The figures for Examples 25, 36 and 37 in the table below show the percentage by weight of the initial mixture, at which the PI/XL mixture (10 parts by weight PI: 20 parts by weight XL) is added. The figure for Example 26 in the table below shows the percentage by weight of the initial mixture, at which the PI/XL mixture (100.7 parts by weight PI: 108 parts by weight XL) is added. The figure for Example 28 in the table below shows the percentage by weight of the initial mixture, at which the PI/XL mixture (I parts by weight PI: 3 parts by weight XL) is added. The figure for Example 29 in the table below shows the percentage by weight of the initial mixture, at which the PI/XL mixture (9 parts by weight PI: 10 parts by weight XL) is added. The figure for Example 30 in the table below shows the percentage by weight of the initial mixture, at which the PI/XL mixture (35 parts by weight PI: 54 parts by weight XL) is added. The figures for Examples 38 to 49 in the table below show the weight of the PI/XL mixture (1 parts by weight PI: 10 parts by weight XL) added.

In each case the mixture was mechanically agitated with a high speed stirrer, to entrain air bubbles in the pre-gel. 50 g of the mixture was then cast on a tray lined with siliconised paper at a coat weight of 1.5 kg/sq.m and was cured in the laboratory by passing at a speed of 7 m/minute three times under ultra-violet (UV) radiation of 80 W/cm from a medium pressure mercury vapour lamp. The resultant cured hydrogel mass had an internal cellular structure, caused by the presence of the air bubbles.

### Example 16 to 49

### Compositions

The ingredients of the compositions of Examples 16 to 49 are shown in the following table:

| | Ex.16 | Ex.17 | Ex.18 | Ex.19 | Ex.20 | Ex.21 | Ex.22 | Ex.23 | Ex.24 |
|---|---|---|---|---|---|---|---|---|---|
| ACMO | 35 | 35 | 35 | 35 | 35 | 35 | 35 | 35 | 35 |
| Water | 65 | 65 | 65 | 65 | 50 | 30 | 20 | 10 | 10 |
| Glycerol | 0 | 0 | 0 | 0 | 15 | 35 | 45 | 55 | 55 |
| PI/XL | 0.1 | 0.2 | 0.3 | 0.4 | 0.4 | 0.4 | 0.4 | 0.4 | 0.4 |
| NaAMPS | 0 | 0 | 0 | 0 | 0 | 0 | 0 | 0 | 4 |
| P65 | 2 | 2 | 2 | 2 | 2 | 2 | 2 | 2 | 2 |

| | Ex.25 | Ex.26 | Ex.27 | Ex.28 | Ex.29 | Ex.30 | Ex.31 | Ex.32 |
|---|---|---|---|---|---|---|---|---|
| ACMO | 35 | 35 | 35 | 35 | 35 | 35 | 20 | 20 |
| Water | 20 | 20 | 20 | 20 | 20 | 20 | 80 | 80 |
| Glycerol | 45 | 45 | 45 | 45 | 45 | 45 | 0 | 0 |
| PI/XL | 0.3 | 0.21 | 0.147 | 0.41 | 0.18 | 0.16 | 0.30 | 0.40 |
| P65 | 2 | 2 | 2 | 2 | 2 | 2 | 2 | 2 |
| | Ex.33 | Ex.34 | Ex.35 | Ex.36 | Ex.37 | Ex.38 | Ex.39 | Ex.40 |
| ACMO | 30 | 30 | 35 | 35 | 35 | 1.5 g | 1.5 g | 2 g |
| Water | 28 | 28 | 20 | 20 | 20 | 0 g | 2 g | 8 g |
| Glycerol | 40 | 40 | 45 | 45 | 45 | 0 g | 0 g | 0 g |
| PI/XL | 0.35 | 0.25 | 0.40 | 0.30 | 0.15 | 0.03 g | 0.03 g | 0.03 g |
| AA | 2 | 2 | 0 | 0 | 0 | 0 g | 0 g | 0 g |
| Salt | 0 | 0 | 0 | 0 | 0 | 10 g | 8 g | 2 g |
| P65 | 2 | 2 | 2 | 2 | 2 | 0.2 g | 0.2 g | 0.2 g |

| | Ex.41 | Ex.42 | Ex.43 | Ex.44 | Ex.45 | Ex.46 | Ex.47 | Ex.48 | Ex.49 |
|---|---|---|---|---|---|---|---|---|---|
| ACMO | 1 g | 1.5 g | 2.5 g | 1.5 g | 1.5 g | 1.5 g | 2 g | 1.5 g | 1.5 g |
| Water | 0 g | 0 g | 0 g | 0 g | 2 g | 0 g | 8 g | 3 g | 2.3 g |
| Glycerol | 0 g | 0.75 g | 1.5 g | 0 g | 0 g | 0 g | 0 g | 0 g | 0 g |
| PI/XL | 0.03 g | 0.03 g | 0.03 g | 0.03 g | 0.03 g | 0.03 g | 0.03 g | 0.03 g | 0.03 g |
| AA | 0.5 g | 0 g | 0.5 g | 0 g | 0 g | 0 g | 0 g | 0 g | 0 g |
| Salt | 10 g | 10 g | 20 g | 10 g | 8 g | 10 g | 2 g | 7 g | 7.7 g |
| P65 | 0.2 g | 0.2 g | 0.1 g | 0.2 g | 0.2 g | 0.2 g | 0.2 g | 0.2 g | 0.2 g |

### Example 50

### Preparative Method and Composition

40.84 g of a 58% aqueous solution of NaAMPS (Lubrizol) were mixed with 25 g of a 79% aqueous solution of acryloyloxyethyltrimethyl ammonium chloride (DMAEA-Q (Kohjin)) and 34.16 g of glycerol for 30 minutes and 3 g of Pluronic P65 (Ciba Geigy). To this mixture 0.19 g of a Daracur 1173 photoinitiator (4 parts) and polyethylene glycol diacrylate (IRR 280, UCB)(20 parts) solution was added and stirred for 30 mintutes. The mixture was mechanically agitated to produce foamed liquid and then coated on to a siliconised polyester backing and passed under a UV lamp. The mixture cured rapidly to produce a gel with good tack and adhesion properties.

### Examples 51 to 57

### Preparative Methods

Non-z-Swelling Foam Structures

The exemplified methods of making z-swelling-restricted porous hydrogels of the present invention involve control of the method of making the foam and the nature of the surfactant or mixtures of surfactants used. For a given surfactant, for example polyoxypropylene-polyoxyethylene block copolymer surfactants such as F68 or P65, available from BASF, the higher the speed of mixing the greater the level of closed cell porous hydrogel relative to open cell. If the mixer speed, is not sufficient then only open cell materials are made.

Blending

1. Foams Containing F68 as Surfactant

A pre-mix of the crosslinker and the photoiniator was made by adding 20 g of Irgacure 280 to 1 g of Daracure 1173. This was stirred in the dark for at least 1 hour. Once made, this mixture can be stored in the dark for several weeks.

The F68 and any P65 (melted) required were weighed out into a dry beaker of appropriate size for foaming. The required amount the Daracure 1173 and Irgacure 280 pre-mix were then added, followed by the monomer, and then glycerol or other humectant(s). The mixture was then stirred in the dark until the F68 surfactant had dissolved. After the surfactant had dissolved the magnetic stirring bar was removed and the mixture foamed using one of the methods described below (as stated in the tables below).

2. Foams Containing P65 as Surfactant

A pre-mix of the Daracure 1173 and Irgacure wass made by adding 20 g of Irgacure 280 to 1 g of Daracure 1173. This was stirred in the dark for at least 1 hour. Once made, this mixture can be stored in the dark for several weeks.

The required amount of Daracure 1173 and Irgacure 280 pre-mix was weighed into a dry vessel of appropriate size for foaming. The required amount of melted P65 surfactant was then added, followed by the monomer and the glycerol (or other humectants). The mixture was then foamed using one of the methods described below (as stated in the tables below).

Foaming

Propellor Method

Approximately 50 g of the polymerisable mixture is weighed into a 100 ml jar. A propeller mixer is then used to stir the mixture at high speed (setting 10 on a RW16 Basic mixer from IKA Labortechnik; this equates to approximately 1200 rpm) for 3 minutes until the mixture is white with the texture of double cream. The mixture will appear smooth and even with no large bubbles on the surface on the mixture. The foamed mixture is poured out and cured with UV light.

Paddle Stirrer at High Speed

Approximately 100 g of polymerisable mixture is weighed into a 600 ml beaker. A paddle stirrer is then used to stir the mixture at high speed (setting 7 on a RW16 Basic mixer from IKA Labortechnik, this equates to approximately 800 rpm) for 3 minutes until the mixture is white with the texture of double cream. The mixture will appear smooth and even, with no large bubbles on the surface on the mixture. The foamed mixture is poured out and cured with UV light.

Paddle Stirrer at Intermediate Speed with Different Surfactant Systems

Approximately 100 g of polymerisable mixture comprising F68 as a surfactant (Examples 51 and 52) is weighed into a 600 ml beaker. A paddle stirrer is then used to stir the mixture at intermediate speed (setting 5 on a RW16 Basic mixer from IKA Labortechnik this equates to 550 rpm) for 3 minutes until the mixture is white with the texture of double cream. The mixture will appear smooth and even, with no large bubbles on the surface on the mixture. The foamed mixture is poured out and cured with UV light.

Approximately 100 g of polymerisable mixture comprising P65 as a surfactant (Example 57) is weighed into a 600 ml beaker. A paddle stirrer is then used to stir the mixture at intermediate speed (setting 5 on a RW16 Basic mixer from IKA Labortechnik, this equates to 550 rpm) for 3 minutes until the mixture is white with the texture of double cream. The mixture will appear white and bubbly; there may be some large bubbles on the surface on the mixture. The foamed mixture is poured out and cured with UV light.

### Examples 51 to 57

### Compositions

| | | EXAMPLE | | |
|---|---|---|---|---|
| | | 51 | 52 | 53 |
| 2-Acrylamido-2-methylpropane sulphonic acid, | g | 36.6 | 37.1 | 37.1 |
| sodium salt | | | | |
| Water | g | 26.5 | 26.9 | 26.9 |
| Glycerol | g | 34.1 | 34.6 | 34.6 |
| Polyoxypropylene-polyoxyethylene block copolymer, | g | 0 | 0 | 0.5 |
| P65 | | | | |
| Polyoxypropylene-polyoxyethylene block copolymer, | g | 2.8 | 1.4 | 0.9 |
| F68 | | | | |
| Daracure 1173 | g/100 g | 0.025 | 0.026 | 0.026 |
| Irgacure 280 | g/100 g | 0.507 | 0.514 | 0.515 |
| Mix Method | | Paddle | Paddle | Paddle |
| Mix Time | Mins | 3 | 3 | 3 |
| | mixer | | | |
| Mix Speed | setting | 5 | 5 | 5 |
| | | EXAMPLE | | |
| | | 54 | 55 | 56 |
| 2-Acrylamido-2-methylpropane sulphonic acid, | g | 37.1 | 38 | 38 |
| sodium salt | | | | |
| Water | g | 26.9 | 27.6 | 27.6 |
| Glycerol | g | 34.6 | 33 | 33 |
| Polyoxypropylene-polyoxyethylene block copolymer, | g | 1 | 1.4 | 1.4 |
| P65 | | | | |
| Polyoxypropylene-polyoxyethylene block copolymer, | g | 0.5 | 0 | 0 |
| F68 | | | | |
| Daracure 1173 | g/100 g | 0.025 | 0.02 | 0.02 |
| Irgacure 280 | g/100 g | 0.507 | 0.538 | 0.538 |
| Mix Method | | Paddle | Paddle | Propellor |
| Mix Time | mins | 3 | 3 | 3 |
| | mixer | | | |
| Mix Speed | setting | 5 | 7 | 10 |

| | | EXAMPLE |
|---|---|---|
| | | 57 |
| 2-Acrylamido-2-methylpropane sulphonic acid, sodium salt | g | 38 |
| Water | g | 27.6 |
| Glycerol | g | 33 |
| Polyoxypropylene-polyoxyethylene block copolymer, P65 | g | 1.4 |
| Polyoxypropylene-polyoxyethylene block copolymer, F68 | g | 0 |
| Daracure 1173 | g/100 g | 0.025 |
| Irgacure 280 | g/100 g | 0.507 |
| Mix Method | | Paddle |
| Mix Time | mins | 3 |
| | mixer | |
| Mix Speed | setting | 2 |

Results and Discussion

### Examples 1 to 6

### Test Results and Discussion

Certain physical parameters of the compositions prepared in Examples 1 to 6 were tested using the test methods described above. The results are shown below (Aw=water activity):

| | | | Cured Foam | |
|---|---|---|---|---|
| | | | Water | Cured Foam |
| | | Foam | Absorption | Water |
| | Pre-Foam | Pre-Cure | Continuous | Absorption |
| | Viscosity | Viscosity | Layer | Porous Layer |
| Example | (mPas) | (mPas) | (microl/s) | (microl/s) |
| 1 | 33 | 324 | 0 | 5 |
| 2 | 28 | 878 | 0.1 | 4 |
| 3 | 40 | 640 | 0 | 25 |
| 4 | 29 | 465 | 5 | 13 |
| 5 | Na | Na | 1 | 4 |
| 6 | Na | Na | 0 | 3 |

| | | | Cured Foam | |
|---|---|---|---|---|
| | Cured Foam | Cured Foam | Viscous | |
| | Elastic | Elastic | Modulus | |
| | modulus @1 | Modulus@100 | @1 (rad/s) | |
| Example | (rad/s) (Pa) | (rad/s) (Pa) | (Pa) | Aw |
| 1 | 8887 | 13730 | 1487 | 0.74 |
| 2 | 8197 | 16666 | 2636 | 0.78 |
| 3 | 1688 | 3305 | 467 | 0.48 |
| 4 | 1567 | 3714 | 535 | 0.48 |
| 5 | 5062 | 10386 | 1383 | 0.46 |
| 6 | 14479 | 99239 | 9698 | 0.27 |

In all of Examples 1 to 15, the foamed hydrogels produced were acceptable gels having good to excellent water uptake rate on the porous side. In the Examples tested (Examples 1 to 6), the foamed hydrogels had acceptable water activity, elastic and viscous moduli for use in the applications described above.

### Examples 16 to 49

### Results and Discussion

Example 16 gave a gel which was clear and colourless, soft and leggy. Example 17 gave a gel which was clear and colourless, a nice soft gel. Example 18 gave a gel which was clear, colourless and tough. Example 19 gave a gel which was clear and colourless, a tough and brittle gel. Example 20 gave a gel which was clear and colourless, tough and slightly tacky. Examples 21 and 22 gave gels which were clear and colourless, tough and tacky. All the above gels were acceptable.

Example 23 gave a gel which was white, hard and brittle and showed syneresis of the glycerol. This gel was unacceptable for use as a bioadhesive. It is believed that this unacceptability may be more generally observed at very high levels of organic plasticiser. However, as shown by Example 24, the problem is surprisingly and effectively overcome by the presence of a small amount of the ionic comonomer (N PS) in the pre-gel. Example 24 gave an acceptable clear, colourless, tough gel.

Example 25 gave a gel which was clear and colourless, soft and tacky. Examples 26 and 27 gave gels which were leggy. Example 28 gave a gel which was clear and colourless, tough, tacky and brittle. Examples 29 and 30 gave clear leggy gels. Example 31 gave a gel which was soft, clear and leggy. Example 32 gave a gel which was clear but brittle. Example 33 gave a gel which was clear and strong. Example 34 gave a gel which was clear but soft. Examples 35 to 37 gave gels which were clear and slightly tacky. Examples 38 to 49 gave acceptable gels, many of which displayed substantial robustness under extremes of temperature and atmospheric dryness. In summary, all of Examples 25 to 49 produced acceptable gels.

### Example 50

### Results and Discussion

The polymerisable mixture cured rapidly to produce a gel with good tack and adhesion properties. The gel has low saline uptake compared to gel made using the same method but replacing the DMAEA-Q with NaAMPS.

### Examples 51 to 57

### Results and Discussion

Examples 51 to 56 all gave foamed porous hydrogels having substantially no z-swelling on exposure to external water. Example 57 showed some degree of z-swelling.

### INDUSTRIAL APPLICABILITY

The present invention makes available porous hydrogels with useful capacity to absorb potentially large quantities of liquids at an acceptable speed for many uses. Moreover, the hydrogels can be made conveniently and efficiently. The process can be such that polymerisation of the polymerisable (pre-gel) mixture is substantially the final processing step in the hydrogel manufacture, with no or only very trivial post-processing of the hydrogel being required. Alternatively, the porosity of the hydrogel can make it attractive to load additional components into the porous structure after initial polymerisation, preferably on the same support arrangement on which the polymerisable mixture was laid down before polymerisation, thereby reducing manufacturing complexity and the risk of contamination through handling.

The present invention has been broadly described without limitation. Variations and modifications as will be readily apparent to those skilled in the art are intended to be covered by the present application and resultant patent(s).

1-39. (canceled)

40. A hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

41. A hydrogel composition according to claim 40, wherein the first portion comprises a porous foam having an internal cellular structure such that the volume ratio of cell void to matrix is greater than about 1:3.

42. A hydrogel composition according to claim 40, wherein the second portion has a volume ratio of cell void to matrix less than about 1:10.

43. A process for the preparation of a porous hydrogel, which comprises polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

44. A process according to claim 43, when used to prepare a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

45. A process according to claim 43, wherein the polymerisable mixture is laid down in sheet or layer form on a suitable support arrangement for the polymerization procedure, whereby the first portion of the polymerisable mixture sits on the second portion.

46. A porous hydrogel composition comprising a flexible plasticised hydrophilic polymer matrix having an internal cellular structure, wherein the hydrophilic polymer is selected from polymers of any of the following monomers:
2-acrylamido-2-methylpropane sulphonic acid or a substituted derivative or salt thereof;
acrylic acid (3-sulphopropyl) ester or a substituted derivative or salt thereof;
a non-ionic monomer containing an alkyl or alkylene or substituted alkyl or alkylene group linked to a carbon-carbon double bond via an amido or alkylamido function;
any mixture of the foregoing with each other or with one or more comonomer;
a monomer/comonomer pair consisting of a first monomer comprising one or more pendant anionic group and a second monomer comprising one or more pendant cationic group; and
any mixture of the said monomer/comonomer pair with any of the foregoing.

47. A porous hydrogel composition according to claim 46, wherein the non-ionic monomer containing an alkyl or alkylene or substituted alkyl or alkylene group linked to a carbon-carbon double bond via an amido or alkylamido function is selected from diacetone acrylamide, a vinyl lactam, an N-alkylated acrylamide, and N,N-dialkylated acrylamide, N-vinyl pyrrolidone, N-acryloyl morpholine, and any mixture thereof.

48. A porous hydrogel composition according to claim 46, wherein, in the monomer/comonomer pair consisting of a first monomer comprising one or more pendant anionic group and a second monomer comprising one or more pendant cationic group, the relative amounts of the said monomers in the pair are such that the anionic groups and the cationic groups are present in essentially equimolar quantities.

49. A porous hydrogel composition according to claim 46, wherein the monomer is selected from 2-acrylamido-2-methylpropane sulphonic acid or a salt thereof, acrylic acid (3-sulphopropyl) ester or a salt thereof, and any mixture thereof.

50. A porous hydrogel composition according to claim 47, wherein the monomer is N-acryloyl morpholine.

51. A process for the preparation of a porous hydrogel composition as defined in claim 46, which comprises polymerising a polymerisable mixture comprising a hydrophilic monomer selected from said monomers and monomer mixtures, wherein the polymerisable mixture includes introduced gas bubbles.

52. A process for the preparation of a porous hydrogel composition, comprising polymerising a polymerisable mixture comprising a hydrophilic monomer and optionally one or more comonomer, wherein the polymerisable mixture includes bubbles consisting predominantly of air, the bubbles having been introduced into the mixture under an atmosphere consisting predominantly of air, and the mixture having been laid down for the said polymerisation after introduction of the bubbles into the polymerisable mixture but before polymerisation.

53. A process according to claim 52, when used for the preparation of a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

54. A process according to claim 52, wherein the polymerisable mixture has a bubble to mixture volume ratio greater than about 1:3.

55. A process according to claim 43, wherein the gassed (foamed) polymerisable mixture is laid down prior to polymerisation in a way which comprises casting the gassed mixture into sheet form.

56. A bioadhesive article adapted to be adhered to skin in use, the article comprising an adhesive for contacting the skin and a substrate supporting the adhesive, wherein the adhesive comprises a bioadhesive porous plasticized hydrophilic polymer having an internal cellular structure.

57. The bioadhesive article according to claim 56, wherein the bioadhesive polymer comprises a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

58. The bioadhesive article according to claim 56, wherein the bioadhesive polymer comprises a hydrogel composition prepared by a process comprises polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

59. A bioadhesive article according to claim 56, wherein the bioadhesive polymer is present in sheet or layer form.

60. A bioadhesive article according to claim 56, wherein the bioadhesive polymer is protected by a release layer, the release layer having been applied to the bioadhesive polymer prior to the polymerization procedure.

61. A process for preparing a bioadhesive article as defined in claim 60, wherein prior to the polymerisation the release layer defines an upper surface of a support arrangement for the polymerisable mixture, and the polymerisable mixture is laid down on said release layer.

62. A wound or burn dressing comprising an absorbent member adapted to contact a wearer's skin in the location of a wound or burn, and a sheet backing member supporting the absorbent member, the sheet backing member including a portion which extends beyond the absorbent member and defines a skin-directed surface which carries a pressure-sensitive adhesive for securement of the dressing to the wearer's skin, wherein the said absorbent member comprises a porous hydrophilic polymer having an internal cellular structure.

63. A wound or burn dressing according to claim 62, wherein the porous hydrophilic polymer comprises a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

64. A wound or burn dressing according to claim 62, wherein the porous hydrophilic polymer is prepared by a process comprising polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

65. A wound or burn dressing according to claim 62, wherein the sheet backing member comprises a foamed polymer, an unfoamed polymer, a woven natural fibres fabric, a non-woven natural fibres fabric, a woven synthetic fibres fabric, a non-woven synthetic fibres fabric, or any combination thereof.

66. A wound or burn dressing according to claim 62, wherein the pressure sensitive adhesive comprises an acrylic-based polymeric pressure-sensitive adhesive; a bioadhesive non-porous hydrogel or gel; or a bioadhesive porous plasticized hydrophilic polymer having an internal cellular structure.

67. A wound or burn dressing according to claim 66, wherein the bioadhesive porous plasticized hydrophilic polymer having an internal cellular structure comprises a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

68. A wound or burn dressing according to claim 66, wherein the bioadhesive porous plasticised hydrophilic polymer having an internal cellular structure comprises a hydrogel composition prepared by a process comprising polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

69. A process for the preparation of a hydrogel composition, which comprises preparing a porous hydrogel composition in sheet or layer form by polymerising a polymerisable mixture on a suitable support arrangement to obtain a porous hydrogel composition in sheet or layer form in which at least the upper face of the sheet or layer is porous, and applying to the porous upper face of the sheet or layer, while the sheet or layer is on the support arrangement on which it was polymerised, a liquid composition comprising the secondary component of the hydrogel composition or a precursor thereof, followed by setting, curing or drying of the secondary component within the porous structure if desired.

70. A process according to claim 69, wherein the application of the liquid composition comprising the secondary component of the hydrogel composition or the precursor thereof takes place on the same day as the polymerisation to form the porous hydrogel material.

71. A process according to claim 69, wherein any subsequent desired setting, curing or drying takes place on the same day as the application of the liquid composition comprising the secondary component of the hydrogel composition or the precursor thereof.

72. A process according to claim 69, when used for the preparation of a hydrogel composition comprising a first portion which comprises a flexible plasticized hydrophilic polymer matrix having an internal cellular structure, and a second portion which comprises a flexible plasticized hydrophilic polymer matrix having relatively continuous internal structure,
in which at least some of the cells contain one or more secondary hydrogel component selected from electrolytes, pH regulators, colorants, chloride sources, bioactive compounds such as antimicrobials, antibiotics, antiseptics, haemostatic agents, wound healing agents, pharmaceuticals and drugs, burn healing agents, skin cooling agents, skin moisturizing agents, and skin warming agents, aroma agents, perfumes, fragrances, scents, polymers, and natural, synthetic and semi-synthetic gel materials.

73. A porous hydrogel material having an internal cellular structure and containing within at least some of the cells one or more secondary hydrogel component selected from electrolytes, pH regulators, colorants, chloride sources, bioactive compounds such as antimicrobials, antibiotics, antiseptics, haemostatic agents, wound healing agents, pharmaceuticals and drugs, burn healing agents, skin cooling agents, skin moisturizing agents, and skin warming agents, aroma agents, perfumes, fragrances, scents, polymers, and natural, synthetic and semi-synthetic gel materials.

74. A porous hydrogel material according to claim 73, wherein the internal cellular structure comprises cell walls formed of a hydrogel composition comprising a first portion which comprises a flexible plasticized hydrophilic polymer matrix having an internal cellular structure, and a second portion which comprises a flexible plasticized hydrophilic polymer matrix having relatively continuous internal structure

75. A porous hydrogel material according to claim 73, wherein the internal cellular structure comprises cell walls formed of a hydrogel composition prepared by a process comprising polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

76. A water-absorbent structure comprising a porous hydrogel portion which comprises a flexible plasticized hydrophilic polymer matrix having a predominantly open-cell internal cellular structure, and a relatively non-porous further portion underlying the porous hydrogel portion, wherein the porous hydrogel portion comprises a sheet or layer of thickness less than about 0.7 mm.

77. A water-absorbent structure according to claim 76, wherein the relatively non-porous further portion underlying the porous hydrogel portion comprises the same hydrogel material as the porous portion.

78. A water-absorbent structure according to claim 76, wherein the relatively non-porous further portion underlying the porous hydrogel portion comprises a different hydrogel material, a non-hydrogel material, or any combination of these materials.

79. A water-absorbent structure according to claim 76, wherein the flexible plasticized hydrophilic polymer matrix having a predominantly open-cell internal cellular structure comprises a hydrogel composition comprising a first portion which comprises a flexible plasticized hydrophilic polymer matrix having an internal cellular structure, and a second portion which comprises a flexible plasticized hydrophilic polymer matrix having relatively continuous internal structure.

80. A water-absorbent structure according to claim 76, wherein the flexible plasticized hydrophilic polymer matrix having a predominantly open-cell internal cellular structure comprises a hydrogel composition prepared by a process comprising polymerising a polymerisable mixture comprising a hydrophilic monomer and optionally one or more comonomer, wherein the polymerisable mixture comprises a first portion including a relatively high concentration of introduced gas bubbles and a second portion including a relatively low concentration of gas bubbles.

81. A process for the preparation of a hydrogel structure comprising a porous hydrogel portion which comprises a flexible plasticised hydrophilic polymer matrix having a predominantly open-cell internal cellular structure, and a relatively non-porous further portion underlying the porous portion, wherein the porous hydrogel portion is in the form of a sheet or layer of thickness less than about 0.7 mm, the process comprising forming by admixture of the ingredients a polymerisable mixture comprising one or more monomer, a curing system for the monomer(s), at least one surfactant and at least one plasticiser, the mixture including introduced gas bubbles, and polymerizing the polymerisable mixture, wherein during the forming of the polymerisable mixture at least some of the ingredients are mixed together using a rotary mixer moving forward at a speed of more than about 500 rpm.

82. A process according to claim 81, when used to prepare a hydrogel composition comprising a first portion which comprises a flexible plasticized hydrophilic polymer matrix having an internal cellular structure, and a second portion which comprises a flexible plasticized hydrophilic polymer matrix having relatively continuous internal structure

83. A process according to claim 81, when used to prepare a water-absorbent structure comprising a porous hydrogel portion which comprises a flexible plasticized hydrophilic polymer matrix having a predominantly open-cell internal cellular structure, and a relatively non-porous further portion underlying the porous hydrogel portion, wherein the porous hydrogel portion comprises a sheet or layer of thickness less than about 0.7 mm.

84. A process according to 43, wherein at least one further monomer or other desired component or components of the hydrogel composition or water-absorbent structure or precursor thereof is added as a liquid to the polymerisable mixture after it has been laid down on a suitable support arrangement and before polymerisation, the conditions being such that the at least one further monomer or other desired component or components or precursor percolates through an upper foam layer of the polymerisable mixture and mixes preferentially into a relatively bubble-free layer of the polymerisable mixture underlying the foam layer.
